(19) United States
(12) Patent Application Publication
(10) Pub. No.: US 2009/0191145 A1
(43) Pub. Date: Jul. 30, 2009

(54) ADHESIVE SYSTEMS CONTAINING POLYISOCYANATE PREPOLYMERS AND ASPARTATE-ESTER CURING AGENTS, PROCESSES FOR PREPARING THE SAME, MEDICAL USES THEREFOR AND DISPENSING SYSTEMS FOR THE SAME

(75) Inventors: Heike Heckroth, Odenthal (DE); Burkhardt Kohler, Zierenberg (DE); Sebastian Dorr, Dusseldorf (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/358,346

(22) Filed: Jan. 23, 2009

(30) Foreign Application Priority Data
Jan. 24, 2008 (EP) 08001290.9

(51) Int. Cl. A61K 31/785; C09J 175/00

(52) U.S. Cl. 424/78.06; 528/84; 525/453

(57) ABSTRACT

Adhesive systems comprising: (A) an isocyanate group-containing prepolymer prepared by reacting: (A1) an aliphatic isocyante; and (A2) a polyol having a number average molecular weight of ≧400 g/mol and 2 to 6 OH groups; and (B) a curing component comprising: (B1) an amino group-containing aspartate ester of the general formula (I);

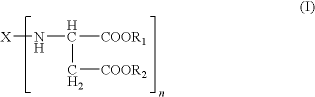

wherein X represents an n-valent organic radical derived from a corresponding n-functional primary amine X(NH₂)ₙ, R₁ and R₂ each independently represent an organic radical having no Zerevitinov active hydrogens and n represents a whole number of at least 2; and (B2) an organic filler having a viscosity of 10 to 6000 mPas at 23° C. measured according to DIN 53019; their use in wound and tissue incision closure, adhesive films comprising the same and dispensing systems therefor.

### BACKGROUND OF THE INVENTION

In recent years, increasing interest has developed in the replacement or complementation of surgical sutures through the use of suitable adhesives. Particularly in the field of plastic surgery, in which particular value is placed on thin, as far as possible invisible scars, adhesives are being increasingly used.

Tissue adhesives must have a number of properties in order to be accepted among surgeons as a substitute for sutures. These include ease of use and an initial viscosity such that the adhesive cannot penetrate into deeper tissue layers or run off. In classical surgery, rapid curing is required, whereas in plastic surgery correction of the adhesive suture should be possible and thus the curing rate should not be too rapid (ca. 5 mins). The adhesive layer should be a flexible, transparent film, which is not degraded in a time period of less than three weeks. The adhesive must be biocompatible and must not display histotoxicity, nor thrombogenicity or potential allergenicity.

Various materials which are used as tissue adhesives are commercially available. These include the cyanoacrylates Dermabond® (octyl 2-cyanoacrylate) and Histoacryl Blue® (butyl cyanoacrylate). However, the rapid curing time and the brittleness of the adhesion site limit their use. Owing to their poor biodegradability, cyanoacrylates are only suitable for external surgical sutures.

As alternatives to the cyanoacrylates, biological adhesives such as peptide-based substances (BioGlue®) or fibrin adhesives (Tissucol) are available. Apart from their high cost, fibrin adhesives are characterized by relatively weak adhesive strength and rapid degradation, so that this is only usable for smaller incisions in untensioned skin.

Isocyanates-containing adhesives are generally all based on an aromatic diisocyanate and a hydrophilic polyol, the isocyanates TDI and MDI preferably being used (e.g., US 2003/0135238, US 2005/0129733). Both can bear electron-withdrawing substituents in order to increase their reactivity (WO-A 03/9323).

Difficulties until now were the low mechanical strength (U.S. Pat. No. 5,156,613), excessively slow curing rate (U.S. Pat. No. 4,806,614), excessively rapid biodegradability (U.S. Pat. No. 6,123,667) and uncontrolled swelling (U.S. Pat. No. 6,265,016).

Only polyurethane prepolymers with a trifunctional or branched structure which are also capable of forming hydrogels are suitable adhesives (e.g., US 2003/0135238). The adhesive must also be capable of forming a covalent bond to the tissue. US 2003/0135238 and US 2005/0129733 describe the synthesis of trifunctional, ethylene oxide-rich TDI- and IPDI- (US 2003/0135238) based prepolymers which react with water or with tissue fluids to give the hydrogel. Sufficiently rapid curing was until now only attained with the use of aromatic isocyanates, which however react with the formation of foam. This results in penetration of the adhesive into the wound and hence in the wound edges being pushed part, which results in poorer healing with increased scarring. In addition, the mechanical strength and the adhesion of the adhesive layer is decreased by the foam formation. In addition, on account of the higher reactivity of the prepolymers, reaction of the isocyanate radicals with the tissue takes place, as a result of which denaturation, recognizable through white coloration of the tissue, often occurs.

As a replacement for the aromatic isocyanates, lysine diisocyanate has been studied, but owing to its low reactivity this reacts only slowly or not at all with tissue (US 2003/0135238).

In order to increase their reactivity, aliphatic isocyanates have been fluorinated (U.S. Pat. No. 5,173,301), however this resulted in spontaneous autopolymerization of the isocyanate.

EP-A 0 482 467 describes the synthesis of a surgical adhesive based on an aliphatic isocyanate (preferably HDI) and a polyethylene glycol (Carbowax 400). Curing takes place on addition of 80 to 100% water and a metal carboxylate (potassium octanoate) as catalyst, during which a foam is formed, which is stabilized with silicone oil.

Systems based on aliphatic isocyanates display only insufficient reactivity and hence an excessively slow curing time. Although the reaction rate could be increased by the use of metal catalysts, as described in EP-A 0 482 467, this resulted in the formation of a foam, with the problems described above.

The fundamental suitability of aspartate esters for the crosslinking of prepolymers is well known in the state of the art in the context of surface coatings and is for example described in EP-A 1 081 171 or DE-A 102 46 708.

European Patent Application No. 07021764.1, unpublished at the priority date of the present specification, has already described wound adhesives based on a combination of hydrophilic polyisocyanate prepolymers and aspartates as curing agents. These systems, however, are in some cases difficult to meter and to apply, since the amount of aspartate needed is small in relation to the prepolymer to be cured. This situation can be improved by pre-extending the aspartate with NCO prepolymer.

### BRIEF SUMMARY OF THE INVENTION

The present invention relates, in general, to novel, rapidly curing adhesives based on hydrophilic polyisocyanate prepolymers for use in surgery.

The present invention provides significantly improved wound adhesives based on a combination of hydrophilic polyisocyanate prepolymers and aspartates as curing agents which include specific fillers. The adhesives according to various embodiments of the present invention provide simplified application without requiring pre-extension of aspartate with NCO prepolymer.

The subject matter of the present invention therefore relates to adhesive systems comprising:

A) isocyanate group-containing prepolymers obtainable from
A1) aliphatic isocyanates and
A2) polyols with number-averaged molecular weights of ≧400 g/mol and average OH group contents of from 2 to 6

and

B) a curing component comprising
B1) amino group-containing aspartate esters of the general formula (I)

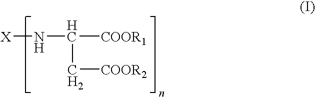

wherein X is an n-valent organic radical, which is obtained by removal of the primary amino groups of an n-functional amine, R₁, R₂ are the same or different organic radicals, which contain no Zerevitinov active hydrogen and n is a whole number of at least 2

and
B2) organic fillers having a viscosity at 23° C. measured to DIN 53019 in the range from 10 to 6000 mPas

and

C) where appropriate, reaction products of isocyanate group-containing prepolymers according to the definition of component A) with aspartate esters according to component B1) and/or organic fillers according to component B2).

For the definition of Zerevitinov active hydrogen, reference is made to Römpp Chemie Lexikon, Georg Thieme Verlag Stuttgart. Preferably, groups with Zerevitinov active hydrogen are understood to mean OH, NH or SH.

In the context of the present invention, tissues are understood to mean associations of cells which consist of cells of the same form and function such as surface tissue (skin), epithelial tissue, myocardial, connective or stromal tissue, muscles, nerves and cartilage. These also include, inter alia, all organs made up of associations of cells such as the liver, kidneys, lungs, heart, etc.

One embodiment of the present invention includes adhesive systems which comprise: (A) an isocyanate group-containing prepolymer prepared by reacting: (A1) an aliphatic isocyante; and (A2) a polyol having a number average molecular weight of ≧400 g/mol and 2 to 6 OH groups; and (B) a curing component comprising: (B1) an amino group-containing aspartate ester of the general formula (I):

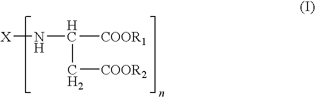

wherein X represents an n-valent organic radical derived from a corresponding n-functional primary amine X(NH₂)ₙ, R₁ and R₂ each independently represent an organic radical having no Zerevitinov active hydrogens and n represents a whole number of at least 2; and (B2) an organic filler having a viscosity of 10 to 6000 mPas at 23° C. measured according to DIN 53019. Additional embodiments of the present invention include human and/or animal tissue adhesives comprising adhesive systems according to any of the various embodiments of the invention. Still other embodiments of the present invention include methods of applying human and/or animal tissue adhesives comprising such adhesive systems to close wounds or surgical incisions.

Another embodiment of the present invention includes processes for producing adhesive systems, which processes comprise: (i) providing (A) an isocyanate group-containing prepolymer prepared by reacting: (A1) an aliphatic isocyante; and (A2) a polyol having a number average molecular weight of ≧400 g/mol and 2 to 6 OH groups; and (B) a curing component comprising: (B1) an amino group-containing aspartate ester of the general formula (I):

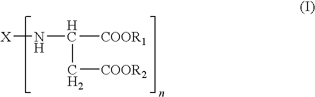

wherein X represents an n-valent organic radical derived from a corresponding n-functional primary amine X(NH₂)ₙ, R₁ and R₂ each independently represent an organic radical having no Zerevitinov active hydrogens and n represents a whole number of at least 2; and (B2) an organic filler having a viscosity of 10 to 6000 in Pas at 23° C. measured according to DIN 53019; and (ii) mixing (A) and (B) in a ratio of NCO-reactive groups to free NCO groups of 1:1.5 to 1:1.

Yet another embodiment of the present invention includes dispensing systems which comprise at least two chambers; wherein a first chamber comprises an amount of (A) an isocyanate group-containing prepolymer prepared by reacting: (A1) an aliphatic isocyante; and (A2) a polyol having a number average molecular weight of ≧400 g/mol and 2 to 6 OH groups; and wherein a second chamber comprises an amount of (B) a curing component comprising: (B1) an amino group-containing aspartate ester of the general formula (I):

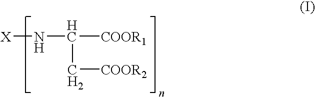

wherein X represents an n-valent organic radical derived from a corresponding n-functional primary amine X(NH₂)ₙ, R₁ and R₂ each independently represent an organic radical having no Zerevitinov active hydrogens and n represents a whole number of at least 2; and (B2) an organic filler having a viscosity of 10 to 6000 mPas at 23° C. measured according to DIN 5301.

### DETAILED DESCRIPTION OF THE INVENTION

As used herein, the singular terms “a” and “the” are synonymous and used interchangeably with “one or more” and “at least one,” unless the language and/or context clearly indicates otherwise. Accordingly, for example, reference to “a polyol” herein or in the appended claims can refer to a single polyol or more than one polyol. Additionally, all numerical values, unless otherwise specifically noted, are understood to be modified by the word “about.”

Isocyanate group-containing prepolymers suitable for use in A) are obtainable by reaction of isocyanates with hydroxy group-containing polyols optionally with the addition of catalysts, auxiliary agents and additives.

As isocyanates in A1), for example, monomeric aliphatic or cycloaliphatic di- or triisocyanates such as 1,4-butylene diisocyanate (BDI), 1,6-hexamethylene diisocyanate (HDI), isophorone diisocyanate (IPDI), 2,2,4- and/or 2,4,4-trimethylhexamethylene diisocyanate, the isomeric bis-(4,4′-isocyanatocyclohexyl)methanes or mixtures thereof of any isomer content, 1,4-cyclo-hexylene diisocyanate, 4-isocyanatomethyl-1,8-octane diisocyaniate (nonane triisocyanate), and alkyl 2,6-diisocyanatohexanoates (lysine diisocyanate) with C1-C8 alkyl groups can be used.

In addition to the aforesaid monomeric isocyanates, higher molecular weight derivatives thereof of uretdione, isocyanurate, urethane, allophanate, biuret, iminooxadiazinedione or oxadiazinetrione structure and mixtures thereof can also be used.

Preferably, isocyanates of the aforesaid nature with exclusively aliphatically or cycloaliphatically bound isocyanate groups or mixtures thereof are used in A1).

The isocyanates or isocyanate mixtures used in A1) preferably have an average NCO group content of from 2 to 4, particularly preferably 2 to 2.6 and quite particularly preferably 2 to 2.4.

In a particularly preferable embodiment, hexamethylene diisocyaniate is used in A1).

For synthesis of the prepolymer, essentially all polyhydroxy compounds with 2 or more OH groups per molecule known per se to a person skilled in the art can be used in A2). These can for example be polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols, polyester polycarbonate polyols or any mixtures thereof one with another.

The polyols used in A2) preferably have an average OH group content of from 3 to 4.

Furthermore, the polyols used in A2) preferably have a number-averaged molecular weight of 400 to 20000 g/mol, particularly preferably 2000 to 10000 g/μmol and quite particularly preferably 4000 to 8500.

Polyether polyols are preferably polyalkylene oxide polyethers based on ethylene oxide and optionally propylene oxide.

These polyether polyols are preferably based on starter molecules with two or more functional groups such as alcohols or amines with two or more functional groups.

Examples of such starters are water (regarded as a diol), ethylene glycol, propylene glycol, butylene glycol, glycerine, TMP, sorbitol, pentaerythritol, triethanolamine, ammonia or ethylenediamine.

Preferred polyalkylene oxide polyethers correspond to those of the aforesaid nature and have a content of ethylene oxide-based units of 50 to 100%, preferably 60 to 90%, based on the overall quantities of alkylene oxide units contained.

Preferred polyester polyols are the polycondensation products, known per se, of di- and optionally tri- and tetraols and di- and optionally tri- and tetracarboxylic acids or hydroxycarboxylic acids or lactones. Instead of the free polycarboxylic acids, the corresponding polycarboxylic acid anhydrides or corresponding polycarboxylate esters of lower alcohols can also be used for the production of the polyesters.

Examples of suitable diols are ethylene glycol, butylene glycol, diethylene glycol, triethylene glycol, polyalkylene glycols such as polyethylene glycol and also 1,2-propaniediol, 1,3-propane-diol, 1,3-butanediol, 1,4-butanediol, 1,6-hexanediol and isomers, neopentyl glycol or neopentyl glycol hydroxypivalate, with 1,6-hexanediol and isomers, 1,4-butanediol, neopentyl glycol and neopentyl glycol hydroxypivalate being preferred. As well as these, polyols such as trimethylol-propane, glycerine, erythritol, pentaerythritol, trimethylolbenzene or trishydroxyethyl isocyanurate can also be used.

As dicarboxylic acids, phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid can be used. The corresponding anhydrides can also be used as the source of acid.

Provided that the average functional group content of the polyol to be esterified is >2, monocarboxylic acids, such as benzoic acid and hexanecarboxylic acid can also be used as well.

Preferred acids are aliphatic or aromatic acids of the aforesaid nature. Particularly preferred are adipic acid, isophthalic acid and phthalic acid.

Examples of hydroxycarboxylic acids, which can also be used as reaction partners in the production of a polyester polyol with terminal hydroxy groups are hydroxycaproic acid, hydroxybutyric acid, hydroxydecanoic acid, hydroxystearic acid and the like. Suitable lactones are caprolactone, butyrolactone and homologues. Caprolactone is preferred.

Likewise, polycarbonates having hydroxy groups, preferably polycarbonate diols, with number-averaged molecular weights Mₙ of 400 to 8000 g/mol, preferably 600 to 3000 g/mol, can be used. These are obtainable by reaction of carboxylic acid derivatives, such as diphenyl carbonate, dimethyl carbonate or phosgene, with polyols, preferably diols.

Possible examples of such diols are ethylene glycol, 1,2- and 1,3-propaniediol, 1,3- and 1,4-butane-diol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, dipropylene glycol, polypropylene glycols, dibutylene glycol, polybutylene glycols, bisphenol A and lactone-modified diols of the aforesaid nature.

Polyether polyols of the aforesaid nature are preferably used for the synthesis of the prepolymer.

For the production of the prepolymer, the compounds of the component A1) are reacted with those of the component A2) preferably with an NCO/OH ratio of 4:1 to 12:1, particularly preferably 8:1, and then the content of unreacted compounds of the component A1) is separated by suitable methods. Thin film distillation is normally used for this, whereby low residual monomer products with residual monomer contents of less than 1 wt. %, preferably less than 0.5 wt. %, quite particularly preferably less than 0.1 wt. %, are obtained.

If necessary, stabilizers such as benzoyl chloride, isophthaloyl chloride, dibutyl phosphate, 3-chloropropionic acid or methyl tosylate can be added during the production process.

The reaction temperature here is 20 to 120° C., preferably 60 to 100° C.

Preferably in formula (I): R₁ and R₂ are alike or different, optionally branched or cyclic organic radicals which contain no Zerevitinov active hydrogen, having 1 to 20, preferably 1 to 10 carbon atoms, more preferably methyl or ethyl groups; n is an integer from 2 to 4; and X is an n-valent organic, optionally branched or cyclic organic, radical having 2 to 20, preferably 5 to 10 carbon atoms, which is obtained by removal of the primary amino groups of an n-valent primary amine.

It is of course possible to use mixtures of two or more aspartic esters, with the consequence that n in formula (I) may also represent a non-integral average value.

The production of the amino group-containing polyaspartate ester B1) can be effected in a known manner by reaction of the corresponding primary at least bifunctional amine X(NH₂), with maleate or fumarate esters of the general formula:

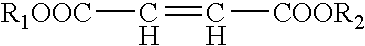

Preferred maleate or fumarate esters are dimethyl maleate, diethyl maleate, dibutyl maleate and the corresponding fumarate esters.

Preferred primary at least bifunctional amines X(NH₂)ₙ, are ethylenediamine, 1,2-diaminopropane, 1,4-diaminobutane, 1,3-diaminopentane, 1,5-diaminopentane, 2-methyl-1,5-diaminopentane, 1,6-diaminohexane, 2,5-diamino-2,5-dimethylhexane, 2,2,4- and/or 2,4,4-trimethyl-1,6-diaminohexane, 1,11-diaminoundecane, 1,12-diaminododecane, 1-amino-3,3,5-trimethyl-5-aminomethyl-cyclohexane, 2,4- and/or 2,6-hexahydrotoluoylenediamine, 2,4′-and/or 4,4′-diamino-dicyclohexylmethane, 3,3′-dimethyl-4,4′-diamino-dicyclohexyl-methane, 2,4,4′-triamino-5-methyl-dicyclohexylmethane and polyether amines with aliphatically bound primary amino groups with a number-averaged molecular weight Mₙ, of 148 to 6000 g/mol.

Particularly preferred primary at least bifunctional amines are 1,3-diaminopentane, 1,5-diaminopentane, 2-methyl-1,5-diaminopentane, 1,6-diaminohexane and 1,13-diamino-4,7,10-trioxatridecane. Most particular preference is given to 2-methyl-1,5-diaminopentane.

In a preferred embodiment of the invention, R₁═R₂=ethyl, X being based on 2-methyl-1,5-diaminopentane as the n-functional amine.

The production of the amino group-containing aspartate ester B1) from the said starting materials can be effected according to U.S. Pat. No. 5,243,012, the entire contents of which are hereby incorporated herein by reference, preferably within the temperature range from 0 to 100° C., the starting materials being used in quantity proportions such that for every primary amino group at least one, preferably exactly one, olefinic double bond is removed, wherein starting materials possibly used in excess can be removed by distillation after the reaction The reaction can be effected neat or in the presence of suitable solvents such as methanol, ethanol, propanol or dioxan or mixtures of such solvents.

The organic liquid fillers used in B2) are preferably not cytotoxic by cytotoxicity measurements in accordance with ISO 10993.

Organic fillers which can be used include polyethylene glycols such as PEG 200 to PEG 600, their monoalkyl and dialkyl ethers such as PEG 500 dimethyl ether, polyether polyols and polyester polyols, polyesters such as Ultramoll, Lanxess GmbH, DE, and also glycerol and its derivatives such as triacetin, Lanxess GmbH, DE, provided that they meet the as-claimed viscosity.

The organic fillers of component B2) are preferably hydroxy- or amino-functional compounds, preferably purely hydroxy-functional compounds. Particular preference is given to polyols. Preferred polyols are polyethers and/or polyester polyols, more preferably polyether polyols.

The preferred organic fillers of component B2) possess preferably average OH group contents of 1.5 to 3, more preferably 1.8 to 2.2, very preferably 2.0.

The preferred organic fillers of component B2) preferably possess repeating units derived from ethylene oxide.

The viscosity of the organic fillers of component B2) is preferably 50 to 4000 mPas at 23° C. as measured in accordance with DIN 53019.

In one preferred embodiment of the invention polyethylene glycols are used as organic fillers of component 132). These glycols preferably have a number-average molecular weight of 100 to 1000 g/mol, more preferably 200 to 400 g/mol.

The weight ratio of B1) to B2) is 1:0.5 to 1:20, preferably 1:0.5 to 1:12.

The weight ratio of component B2 relative to the total amount of the mixture of B1, B2 and A is preferably 1 to 60%.

In order to further reduce the mean equivalent weight of the compounds used overall for prepolymer crosslinking, based on the NCO-reactive groups, in addition to the compounds used in B1) and B2), it is also possible to produce the amino or hydroxyl group-containing reaction products of isocyanate group-containing prepolymers with aspartate esters and/or organic fillers B2), provided that the latter contain amino or hydroxyl groups, in a separate prereaction and then to use these reaction products as a higher molecular weight curing component C).

Preferably, ratios of isocyanate-reactive groups to isocyanate groups of between 50 to 1 and 1.5 to 1, particularly preferably between 15 to 1 and 4 to 1, are used for the pre-extension.

Here, the isocyanate group-containing prepolymer to be used for this can correspond to that of the component A) or else be constituted differently from the components listed as possible components of the isocyanate group-containing prepolymers in the context of this application.

The advantage of this modification by pre-extension is that the equivalent weight and equivalent volume of the curing agent component is modifiable within a clear range. As a result, commercially available 2-chamber dispensing systems can be used for application, in order to obtain an adhesive system which with current chamber volume ratios can be adjusted to the desired ratio of NCO-reactive groups to NCO groups.

The 2-component adhesive systems according to the invention are obtained by mixing of the prepolymer with the curing components B) and/or C). The ratio of NCO-reactive NH groups to free NCO groups is preferably 1:1.5 to 1:1, particularly preferably 1:1.

Directly after mixing together of the individual components, the 2-component adhesive systems according to the invention preferably have a shear viscosity at 23° C. of 1000 to 10 000 mPas, particularly preferably 1000 to 8000 mPas and quite particularly preferably 1000 to 4000 mPas.

At 23° C., the rate until complete crosslinking and curing of the adhesive is attained is typically 30 secs to 10 mins, preferably 1 min to 8 min, particularly preferably 1 min to 5 mins.

A further subject of the invention is adhesive films obtainable from the adhesive systems according to the invention and laminated parts produced therefrom.

In a preferred embodiment, the 2-component adhesive systems according to the invention are used as tissue adhesives for the closure of wounds in associations of human or animal cells, so that clamping or suturing for closure can to a very large extent be dispensed with.

The tissue adhesives according to the invention can be used both in vivo and also in vitro, with use in vivo, for example for wound treatment after accidents or operations, being preferred.

Hence a process for the closure or binding of cellular tissues, characterized in that the 2-component adhesive systems according to the invention are used, is also an object of the present invention.

Likewise a subject of the invention is the use of such 2-component adhesive systems for the production of an agent for the closure or binding of cellular tissues and the 2-chamber dispensing systems containing the components of the adhesive system fundamental to the invention which are necessary for its application.

The invention will now be described in further detail with reference to the following non-limiting examples.

### EXAMPLES

Unless otherwise stated, all percentages quoted are based on weight. As a tissue, beef or pork meat was used for in vitro adhesion. In each case, two pieces of meat (1=4 cm, h=0.3 cm, b=1 cm) were painted at the ends over a 1 cm width with the adhesive and glued overlapping. The stability of the adhesive layer was in each case tested by pulling. PEG=polyethylene glycol

### Example 1

### Prepolymer A

465 g of HDI and 2.35 g of benzoyl chloride were placed in a 11 four-necked flask. 931.8 g of a polyether with an ethylene oxide content of 63% and a propylene oxide content of 37% (each based on the total alkylene oxide content) started with TMP (3-functional) were added within 2 hrs at 80° C. and then stirred for a further hour. Next, the excess HDI was distilled off by thin film distillation at 130° C. and 0.1 mm Hg. 980 g (71%) of the prepolymer with an NCO content of 2.53% were obtained. The residual monomer content was <0.03% HDI.

### Example 2

### Aspartate B

1 mol of 2-methyl-1,5-diaminopentane was slowly added dropwise to 2 mols of diethyl maleate under a nitrogen atmosphere, so that the reaction temperature did not exceed 60° C. The mixture was then heated at 60° C. until diethyl maleate was no longer detectable in the reaction mixture, The product was purified by distillation.

### Example 2a

### Aspartate Component Partially Pre-Extended with Isocyanate Group-Containing Prepolymer

1000 g of HDI (hexamethylene diisocyanate), 1 g of benzoyl chloride and 1 g of methyl para-toluenesulphonate were placed with stirring in a 41 four-necked flask. 1000 g of a bifunctional polypropylene glycol polyether with an average molecular weight of 2000 g/mol were added within 3 hours at 80° C. and then stirred for a further hour. The excess HDI was then distilled off by thin film distillation at 130° C. and 0.1 torr. The prepolymer obtained has an NCO content of 3.7%.

200 g of the prepolymer were fed with stirring at room temperature into 291 g of the aspartate B) from 2-methyl-1,5-diaminopentane in a 11 four-necked flask. This was stirred for a further two hours, until isocyanate groups were no longer detectable by IR spectroscopy. The product obtained had a viscosity of 3740 mPas and an NH equivalent weight of 460 g/eq.

### Tissue Bonding Examples:

### Example 3a

### In Vitro Bonding of Muscular Tissue

1 g of the pre-extended aspartate from Example 2a was charged to the 1 ml capacity chamber of a commercial 2-component injection system. The second chamber, with a capacity of 4 ml, was filled with 4 g of prepolymer A. By downward pressure on the piston, the components were pressed through a top-mounted static mixer with corresponding applicator and the mixture was applied thinly to the tissue. A strong bond occurred within 2 minutes. The sections of tissue could not be separated from one another by tension without fibre tearing. In the case of application to the surface of a tissue, complete curing took place within 3 minutes, with formation of a transparent film.

### Example 3b

### In Vitro Bonding of Skin

The mixture from Example 3a was applied to an area measuring 2×2 cm on the shaved back of a domestic pig, and the adhesive behaviour was observed over a period of one week. Curing to a transparent film took place within 3 minutes. Even after a week the film showed no peeling or change.

### Example 4a

### In Vitro Bonding of Muscular Tissue

0.45 g of PEG 200 (60 mPas/20° C.) were mixed thoroughly with 0.55 g of aspartate B and the mixture was applied with 4 g of the prepolymer A as described in Example 3a. Curing with a strong adhesion joined therewith had taken place within 2 minutes. The sections of tissue could not be separated from one another by tension without fibre tearing. In the case of application to the surface of the tissue, complete curing took place within 3 minutes, with formation of a transparent film.

### Example 4b

### In Vitro Bonding, of Skin

The mixture from Example 4a was applied to an area measuring 2×2 cm on the shaved back of a domestic pig, and the adhesive behaviour was observed over a period of one week, Curing to a transparent film took place within 3 minutes. Even after a week the film showed no peeling or change.

### Comparative Example 5

### In Vitro Bonding of Skin

0.55 g of aspartate B was mixed thoroughly with 4 g of prepolymer A and the mixture was applied to an area measuring 2×2 cm on the shaved back of a domestic pig. The adhesive behaviour was observed over a period of one week. Curing to a transparent film took place within 3 minutes. After 4 days the film showed slight peeling at the edges. In the case of the corresponding in vitro tissue bond, curing with strong adhesion took place within 2 minutes. The sections of tissue could not be separated from one another by tension without fibre tearing.

### Example 6

### In Vitro Bonding of Muscular Tissue

4 g of prepolymer A were stirred thoroughly with a mixture of 6 g of PEG 200 (60 mPas/20° C.) and 0.55 g of aspartate B in a beaker. Immediately thereafter the reaction mixture was applied thinly to the tissue to be bonded. Curing with a strong adhesion joined therewith had taken place within 2 minutes. The sections of tissue could not be separated from one another by tension without fibre tearing. In the case of application to the surface of the tissue, complete curing took place within 3 minutes, with formation of a transparent film.

### Example 7

### In Vitro Bonding of Muscular Tissue

4 g of prepolymer A were stirred thoroughly with a mixture of 12 g of PEG 200 (60 mPas/20° C.) and 0.55 g of aspartate B in a beaker and the mixture was applied thinly to the tissue to be bonded. After 2 minutes a moderate adhesion had occurred. The sections of tissue could be separated from one another by tension without damage.

### Example 8

### In Vitro Bonding of Muscular Tissue

4 g of prepolymer A were stirred thoroughly with a mixture of 18 g of PEG 200 (60 mPas/20° C.) and 0.55 g of aspartate B in a beaker and the mixture was applied thinly to the tissue to be bonded. After 2 minutes only weak adhesion between the two sections of tissue had taken place.

### Example 9

### In Vitro Bonding of Muscular Tissue

0.45 g of PEG 400 (120 mPas/20° C.) were mixed thoroughly with 0.55 g of aspartate B and the mixture was applied with 4 g of the prepolymer A as described in Example 3a. After 2 minutes effective adhesion had taken place. The sections of tissue could not be separated from one another by tension without fibre tearing. In the case of application to the surface of a tissue, complete curing took place within 10 minutes, with formation of a transparent film.

### Example 10

### In Vitro Bonding of Muscular Tissue

4 g of prepolymer A were stirred thoroughly with a mixture of 3.45 g of PEG 400 (120 mPas/20° C.) and 0.55 g of aspartate B in a beaker and the mixture was applied thinly to the tissue to be bonded. After 2 minutes a moderate adhesion had occurred. The sections of tissue could be separated from one another by tension without damage. In the case of application to the surface of a tissue, complete curing took place within 10 minutes, with formation of a transparent film.

### Example 11

### In Vitro Bonding of Muscular Tissue

0.45 g of PEG 600 (180 mPas/25° C.) were mixed thoroughly with 0.55 g of aspartate B and the mixture was applied with 4 g of the prepolymer A as described in Example 3a. After 2 minutes effective adhesion had taken place. The sections of tissue could be separated from one another by tension with slight fibre damage. In the case of application to the surface of the tissue, complete curing took place within 10 minutes, with formation of a transparent film.

### Example 12

### In Vitro Bonding of Muscular Tissue

4 g of prepolymer A were stirred thoroughly with a mixture of 3.45 g of PEG 600 (180 mPas/25° C.) and 0.55 g of aspartate B in a beaker and the mixture was applied thinly to the tissue to be bonded, After 2 minutes a moderate adhesion had occurred. The sections of tissue could be separated from one another by tension without fibre damage. In the case of application to the surface of the tissue, complete curing took place within 10 minutes, with formation of a transparent film.

### Example 13

### In Vitro Bonding of Muscular Tissue

4 g of prepolymer A were stirred thoroughly with a mixture of 6 g of PEG 600 (180 mPas/25° C.) and 0.55 g of aspartate B in a beaker and the mixture was applied thinly to the tissue to be bonded, After 3 minutes a slight adhesion had occurred. The sections of tissue could be separated from one another by tension without fibre damage. In the case of application to the surface of the tissue, complete curing took place within 10 minutes, with formation of a transparent film.

### Example 14

### In Vitro Bonding of Muscular Tissue

4 g of prepolymer A were stirred thoroughly with a mixture of 0.55 g of aspartate B and 3.45 g of a polyether with a molecular weight of 218 and a propylene oxide fraction of 65% and a functionality of 2 (80 mPas/20° C.) in a beaker and the mixture was applied thinly to the tissue to be bonded, After 2 minutes a good adhesion had occurred. The sections of tissue could not be separated from one another by tension without fibre damage. In the case of application to the surface of the tissue or to skin, complete curing took place within a period of 3 minutes, with formation of a transparent film.

### Comparative Examples Relating to the In Vitro Bonding of Muscular Tissue:

### Example 15

4 g of prepolymer A were stirred thoroughly with a mixture of 0.55 g of aspartate B and 3.45 g of a polyester polyol with an ethylene oxide fraction of 52% and a propylene oxide fraction of 35% and a functionality of 3 (3460 mPas/25° C.) in a beaker and the mixture was applied thinly to the tissue to be bonded. After 3 minutes a moderate, after 6 minutes a good adhesion had occurred. The sections of tissue could be separated from one another by tension with slight fibre damage. In the case of application to the surface of the tissue or to skin, complete curing did not occur within a period of 10 minutes.

### Example 16

4 g of prepolymer A were stirred thoroughly with a mixture of 0.55 g of aspartate B and 3.45 g of a polyether of molecular weight 3005 with an ethylene oxide fraction of 55% and a propylene oxide fraction of 45% and a functionality of 3 (550 mPas/25° C.) in a beaker and the mixture was applied thinly to the tissue to be bonded. After 3 minutes a strong bond had taken place. The sections of tissue could not be separated from one another by tension without fibre damage. In the case of application to the surface of the tissue or to skin, complete curing did not occur within a period of 10 minutes.

### Example 17

4 g of prepolymer A were stirred thoroughly with a mixture of 055 g of aspartate B and 3.45 g of a polyether of molecular weight 673 with a propylene oxide fraction of 3.6%, an ethylene oxide fraction of 96.4% and a functionality of 3 (700 mPas/25° C.) in a beaker and the mixture was applied thinly to the tissue to be bonded. After 2 minutes a good bond had taken place. The sections of tissue could not be separated from one another by tension without fibre damage. In the case of application to the surface of the tissue or to skin, complete curing did not occur within a period of 5 minutes.

### Example 18

4 g of prepolymer A were stirred thoroughly with a mixture of 0.55 g of aspartate B and 3.45 g of a polyether of molecular weight 4549 with a propylene oxide fraction of 27.3%, an ethylene oxide fraction of 72.7% and a functionality of 3 (1070 mPas/25° C.) in a beaker and the mixture was applied thinly to the tissue to be bonded. After 2 minutes a moderate bond had taken place. The sections of tissue could not be separated from one another by tension without fibre damage. In the case of application to the surface of the tissue or to skin, complete curing did not occur within a period of 10 minutes.

### Example 19

### In Vitro Bonding of Muscular Tissue

4 g of prepolymer A were stirred thoroughly with a mixture of 0.45 g of PEG 500 dimethyl ether (19 mPas/25° C.) and 0.55 g of aspartate B in a beaker and the mixture was applied thinly to the tissue to be bonded. Strong adhesion had occurred after 2 minutes. The sections of tissue could not be separated from one another by tension without fibre damage. In the case of application to the surface of the tissue, complete curing took place within 5 minutes, with formation of a transparent film.

### Example 20

### In Vitro Bonding of Muscular Tissue

4 g of prepolymer A were stirred thoroughly with a mixture of 3.45 g of PEG 500 dimethyl ether (19 mPas/25° C.) and 0.55 g of aspartate B in a beaker and the mixture was applied thinly to the tissue to be bonded. Only weak adhesion had occurred after 5 minutes. In the case of application to the surface of the tissue there was no curing within 10 minutes.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. An adhesive system comprising:
(A) an isocyanate group-containing prepolymer prepared by reacting: (A1) an aliphatic isocyante; and (A2) a polyol having a number average molecular weight of ≧400 g/mol and 2 to 6 OH groups; and
(B) a curing component comprising: (B1) an amino group-containing aspartate ester of the general formula (I): 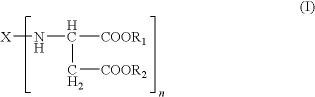
wherein X represents an n-valent organic radical derived from a corresponding n-functional primary amine X(NH₂)ₙ, R₁ and R₂ each independently represent an organic radical having no Zerevitinov active hydrogens and n represents a whole number of at least 2; and (B2) an organic filler having a viscosity of 10 to 6000 mPas at 23° C. measured according to DIN 53019.

2. The adhesive system according to claim 1, further comprising (C) a reaction product of the isocyanate group-containing prepolymer (A) and the curing component (B).

3. The adhesive system according to claim 1, wherein the polyol (A2) has a number average molecular weight of 4000 to 8500 g/mol.

4. The adhesive system according to claim 1, wherein the polyol (A2) comprises a polyalkylene polyether.

5. The adhesive system according to claim 1, wherein the organic filler (B2) comprises a polyether polyol.

6. A human or animal tissue adhesive comprising the adhesive system according to claim 1.

7. A process for producing an adhesive system, the process comprising: (i) providing (A) an isocyanate group-containing prepolymer prepared by reacting: (A1) an aliphatic isocyante; and (A2) a polyol having a number average molecular weight of ≧400 g/mol and 2 to 6 OH groups; and (B) a curing component comprising: (B1) an amino group-containing aspartate ester of the general formula (I): 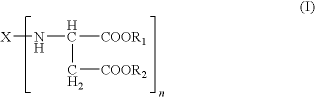
wherein X represents an n-valent organic radical derived from a corresponding n-functional primary amine X(NH₂)ₙ, R₁ and R₂ each independently represent an organic radical having no Zerevitinov active hydrogens and n represents a whole number of at least 2; and (B2) an organic filler having a viscosity of 10 to 6000 mPas at 23° C. measured according to DIN 53019; and (ii) mixing (A) and (B) in a ratio of NCO-reactive groups to free NCO groups of 1:1.5 to 1:1.

8. The process according to claim 7, further comprising providing (C) a reaction product of the isocyanate group-containing prepolymer (A) and the curing component (B); and mixing (C) with (A) and (B).

9. An adhesive system prepared by the process according to claim 7.

10. An adhesive system prepared by the process according to claim 8.

11. A method comprising providing a cellular tissue substrate opening to be closed, and applying the adhesive system according to claim 1 to the cellular tissue substrate such that the opening is closed.

12. An adhesive film comprising the adhesive system according to claim 1.

13. A dispensing system comprising at least two chambers; wherein a first chamber comprises an amount of (A) an isocyanate group-containing prepolymer prepared by reacting: (A1) an aliphatic isocyante; and (A2) a polyol having a number average molecular weight of ≧400 g/mol and 2 to 6 OH groups; and wherein a second chamber comprises an amount of (B) a curing component comprising (B1) an amino group-containing aspartate ester of the general formula (I): 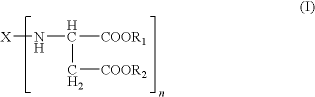
wherein X represents an n-valent organic radical derived from a corresponding n-functional primary amine X(NH₂)ₙ, R₁ and R₂ each independently represent an organic radical having no Zerevitinov active hydrogens and n represents a whole number of at least 2; and (B2) an organic filler having a viscosity of 10 to 6000 mPas at 23° C. measured according to DIN 53019.

14. The dispensing system according to claim 13, further comprising a third chamber, wherein the third chamber comprises an amount of (C) a reaction product of the isocyanate group-containing prepolymer (A) and the curing component (B).
